(19) United States
(12) Patent Application Publication
(10) Pub. No.: US 2023/0022803 A1
(43) Pub. Date: Jan. 26, 2023

(54) NOVEL CRYSTALLINE FORM OF GPR40 AGONIST

(71) Applicant: ILDONG Pharmaceutical Co., Ltd., Seoul (KR)

(72) Inventors: Kiyong LEE, Hwaseong-si (KR); Jisook KWON, Hwseong-si (KR); Hyeran YANG, Hwaseong-si (KR); Hyeongjun LEE, Hwaseong-si (KR)

(21) Appl. No.: 17/358,319

(22) Filed: Jun. 25, 2021

(51) Int. Cl. A61K 31/443; C07D 405/12

(52) U.S. Cl. CPC A61K 31/443; C07D 405/12; C07B 2200/13

(57) ABSTRACT

The present invention relates to a novel crystalline form of Compound 1, a pharmaceutical composition comprising the same, and a method of using the same.

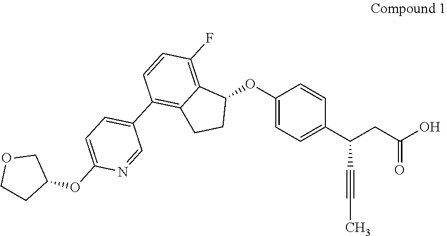 [SMILES: CC#C[C@@H](CC(=O)O)c1ccc(O[C@@H]2CCc3c(-c4ccc(O[C@@H]5CCOC5)nc4)ccc(F)c32)cc1]

### TECHNICAL FIELD

The present invention relates to a novel crystallin form (Form 1) of a G-protein coupled receptor 40 (GPR40) agonist. More particularly, the present invention relates to a novel crystallin form (Form 1) of a GPR40 agonist that has a relatively rapid dissolution rate in aqueous media, to methods of preparing the crystalline form, to pharmaceutical compositions comprising the crystalline form, to methods for treatment of GPR40-mediated diseases, conditions and/or disorders, including conditions and disorders associated with diabetes.

### BACKGROUND ART

Type 2 diabetes mellitus (T2DM), covering up to 95% of the total diabetic patients, is an acquired disease in which environmental factors cause somatic cells to become insulin-resistant, which in terms disables effective absorption of blood glucose. Chronic rise in blood glucose level caused by insulin abnormality leads to serious complications, including obesity, neuralgia, diabetic retinopathy, nephropathy, cardiovascular diseases and dyslipidemia.

G-protein coupled receptor 40 (GPR40), a seven-transmembrane protein, is a type of GPCR of the rhodopsin family, and is primarily expressed in β-cells of pancreatic islets. Since its primary ligands are medium-to-long change fatty acids, the receptor is also known as Free fatty acid receptor 1 (FFAR1). The mechanism of pancreatic β-cell's insulin secretion through GPR40 is mainly determined by either ligands or GPR40 agonists that bind to the receptor. When binding activates the receptor, primary signaling pathway for insulin secretion is promoted through Gα_{q/11}, which is a type of subunits of GPCR. Then, the pathway hydrolyzes cell membrane phospholipids through Phospholipase C (PLC) to produce Diacyglyceral (DAG) and Inositol trisphosphate (IP₃), which subsequently activate Protein Kinase D1 (PKD1) to induce F-actin protein modification, and Calcium ion secretion to ultimately induce insulin secretion.

The mechanism that GPR40 activation induces insulin secretion with blood glucose-dependent manner was proven through experiments using rodent models. (Diabetes, 2007, 56, 1087-1094; Diabetes, 2009, 58, 1067-1076). Such blood glucose-dependent mechanism of insulin secretion has no risk of hypothermia, which makes GPR40 an attractive target for novel drug development. In addition, GPR40 is involved in maintaining pancreatic β-cell survival through regulation of PIX-1 and BCL2, which also results in sustaining of efficacy even in a long-term treatment (BMC Cell Biol., 2014, 15, 24). Furthermore, since the distribution of GPR40 expression is relatively limited, there is low risk of adverse effects in other organs, and improving blood-glucose homeostasis through GPR40 activation is potentially involved in other metabolic disorders including obesity and hypertension. Due to such advantages, efforts have been made to discover novel GPR40 agonists for therapeutic purposes, as disclosed in, for example, U.S. Pat. Nos. 9,856,245, 9,908,873, 9,790,198, and U.S. Application Publication No. 2020/0223833, which are incorporated herein by reference. U.S. Application Publication No. 2020/0223833 discloses compounds having GPR40 agonistic activity. As an example of the compounds, it discloses (S)-3-(4-(((R)-7-Fluoro-4-(6-(((R)-tetrahydrofuran-3-yl)oxy)pyridin-3-yl)-2,3-dihydro-1H-inden-1-yl)oxy)phenyl)hex-4-ynoic acid, but it does not disclose a crystalline form of the compound that has properties desired for therapeutic application including a relatively rapid dissolution rate in aqueous media, a relatively high thermal stability, and the like.

### SUMMARY

In one aspect, the present invention provides a crystalline form of Compound 1, having an X-ray powder diffraction pattern comprising peaks with degrees two-theta positions of about 8.2390, 11.6804, 15.1104, 16.5082 and 20.9169, wherein each peak of degree two-theta has a margin of error of ±0.2.

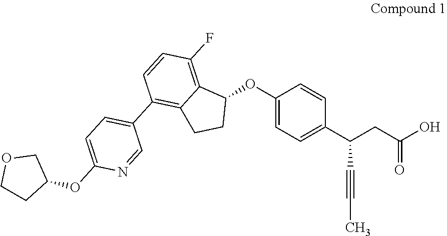 [SMILES: CC#C[C@@H](CC(=O)O)c1ccc(O[C@@H]2CCc3c(-c4ccc(O[C@@H]5CCOC5)nc4)ccc(F)c32)cc1]

In some embodiments, the crystalline form Compound 1 has an X-ray powder diffraction pattern comprising five or more peaks with degrees two-theta positions selected from the group consisting of h degrees two-theta positions selected from the group consisting of 4.2721, 8.2390, 8.5441, 9.0268, 11.6804, 12.8279, 13.3227, 15.1104, 16.0874, 16.2609, 16.5082, 16.6950, 17.3649, 18.1220, 18.5187, 18.9074, 19.1174, 19.2305, 19.9625, 20.6590, 20.9169, 21.4576, 21.9189, 22.1679, 22.7467, 22.8806, 23.1588, 23.4796, 23.6577, 24.1282, 24.3485, 24.9581, 25.1935, 25.5077, 26.2001, 26.5042, 27.2953, 27.3795, 28.1568, 28.7195, 29.1545, 29.8201, 30.3630, 30.9912, 31.3151, 32.1743, 32.4060, 32.8746, 33.6575, 33.9219, 34.3338, 34.6532, 35.3607, 36.0311, 36.4072, 36.7779, 37.7329, 38.1170, 38.7922, 39.3418, 40.4170, 40.8055, 41.5236, 42.3929, and 43.3903, wherein each peak of degree two-theta has a margin of error of ±0.2.

In some embodiments, the crystalline form Compound 1 has an X-ray powder diffraction pattern that is identical or similar to that of FIG. 1.

In some embodiments, the crystalline form Compound 1 has a differential scanning calorimetry thermogram exhibiting an endotherm with an onset of about 136° C.

In some embodiments, the crystalline form Compound 1 has a differential scanning calorimetry thermogram exhibiting an endotherm with an onset of 136.47° C.

In some embodiments, the crystalline form Compound 1 has a differential scanning calorimetry thermogram that is identical or similar to that of FIGS. 2A and 2B.

In another aspect, the present invention provides a solid or semi-solid pharmaceutical dosage form comprising a crystalline form Compound 1 according to an embodiment of the present invention and a pharmaceutically acceptable excipient.

In still another aspect, the present invention provides a pharmaceutical composition comprising a crystalline form Compound 1 according to an embodiment of the present invention and a pharmaceutically acceptable excipient.

In a further aspect, the present invention provides a method for treating a disease, disorder, or condition associated with GPR40, the method comprising administering to a subject a therapeutically effective amount of a pharmaceutical composition comprising a crystalline form Compound 1 according to an embodiment of the present invention and a pharmaceutically acceptable excipient.

In some embodiments, the disease, disorder, or condition may be selected at least one from the group consisting of obesity, type 2 diabetes, incompatible glucose tolerance, insulin resistance, hyperglycemia, hyperlipidemia, hypertriglyceridemia, and hypercholesterolemia.

In some embodiments, one or more additional active or therapeutic agents are administered to the subject with the above-described pharmaceutical composition.

The above and other aspects and embodiments will be described in detail below.

### BRIEF DESCRIPTION OF DRAWINGS

FIG. 1 illustrates an X-ray powder diffraction pattern of a crystalline form Compound 1 in accordance with an embodiment of the present invention.

FIGS. 2A and 2B illustrate the results of TGA and DSC analyses, respectively, of a crystalline form Compound 1 in accordance with an embodiment of the present invention.

FIGS. 3A to 3D illustrate polarized optical microscopy images of a crystalline form Compound 1 in accordance with an embodiment of the present invention.

FIG. 4 illustrates the result of an HPLC analysis of a crystalline form Compound 1 in accordance with an embodiment of the present invention.

FIG. 5 illustrates a solution ¹H-NMR spectrum of a crystalline form Compound 1 in accordance with an embodiment of the present invention.

FIG. 6 illustrates the result of a GVS analysis of a crystalline form Compound 1 in accordance with an embodiment of the present invention.

### DETAILED DESCRIPTION

As used herein and in the appended claims, the singular forms “a,” “and,” and “the” include plural referents unless the context clearly dictates otherwise. Thus, for example, reference to “an agent” includes a plurality of such agents, and reference to “a compound” or “the compound includes reference to one or more compounds and equivalents thereof (e.g., plurality of compounds) known to those skilled in the art, and so forth. The term “about” when referring to a number or a numerical range means that the number or numerical range referred to is an approximation within experimental variability (or within statistical experimental error), and thus the number or numerical range may vary between 1% and 20% of the stated number or numerical range.

The term “crystalline,” as used herein, means having a regularly repeating arrangement of molecules or external face planes.

The term “mixture,” as used herein, means a combination of at least two substances, in which one substance may be completely soluble, partially soluble or essentially insoluble in the other substance.

The term “solvent,” as used herein, means a substance, preferably a liquid or a miscible, partially miscible or immiscible mixture of two or more liquids, which is capable of completely dissolving, partially dissolving, dispersing or partially dispersing another substance, preferably a solid or a mixture of solids.

As used herein, the term “pharmaceutically acceptable” refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

As used herein, the term “pharmaceutically acceptable salt” refers to a formulation of a compound that does not cause significant irritation to an organism to which it is administered and does not abrogate the biological activity and properties of the compounds described herein.

Pharmaceutically acceptable salt forms may include pharmaceutically acceptable acidic/anionic or basic/cationic salts (UK Journal of Pharmaceutical and Biosciences Vol. 2(4), 01-04, 2014, which is incorporated herein by reference). Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-arginine, ammonium, ethanolamine, piperazine, and triethanolamine salts.

A pharmaceutically acceptable acid addition salt of a compound of the invention may be prepared by methods known in the art and may be formed by reaction of the free base form of the compound with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, and hexanoic acid. A pharmaceutically acceptable acid addition salt can comprise or be, for example, a hydrobromide, hydrochloride, sulfate, bisulfate, nitrate, phosphate, succinate, maleate, formarate, acetate, propionate, fumarate, citrate, tartrate, lactate, benzoate, carbonate, benzathine, chloroprocaine, choline, histidine, meglumine, meglumine, procaine, triethylamine, besylate, decanoate, ethylenediamine, salicylate, glutamate, aspartate, p-toluenesulfonate, benzenesulfonate, methanesulfonate, ethanesulfonate, naphthalenesulfonate (e.g., 2-naphthalenesulfonate), and hexanoate salt.

A pharmaceutically acceptable base addition salt of a compound of the invention may also be prepared by methods known in the art and may be formed by reaction of the free base form of the compound with a suitable inorganic or organic base including, but not limited to, hydroxide or other salt of sodium, potassium, lithium, ammonium, calcium, magnesium, iron, zinc, copper, manganese, aluminum, glycolate, hydrabamine, methylbromide, methylnitrate, octanoate, oleate, and the like.

A free acid or free base form of a compound of the invention may be prepared by methods known in the art (e.g., for further details see L. D. Bigley, S. M. Berg, D. C. Monkhouse, in “Encyclopedia of Pharmaceutical Technology”. Eds, J. Swarbrick and J. C. Boylam, Vol 13, Marcel Dekker, Inc., 1995, pp. 453-499, which is incorporated herein by reference). For example, a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Prodrug derivatives of the compounds of the invention may be prepared by methods known to those of ordinary skill in the art (e.g., for further details see Saulnier et al., Bioorg. Med. Chem. Letters, 1994, 4, 1985, which is incorporated herein by reference). Protected derivatives of the compounds of the invention may be prepared by means known to those of ordinary skill in the art. A detailed description of techniques applicable to the creation of protecting groups and their removal can be found in T. W. Greene, “Protecting Groups in Organic Chemistry,” 3rd edition, John Wiley and Sons, Inc., 1999 and “Design of Prodrugs”, ed. 11. Bundgaard, Elsevier, 1985, which are incorporated herein by reference.

The compounds of the invention may be prepared as stereoisomers. Where the compounds of the invention have at least one chiral center, they may exist as enantiomers. Where the compounds possess two or more chiral centers, they may exist as diastereomers. The compounds of the invention may be prepared as racemic mixtures. Alternatively, the compounds of the invention may be prepared as their individual enantiomers or diastereomers by reaction of a racemic mixture of the compound with an optically active resolving agent to form a pair of diastereo-isomeric compounds, separating the diastereomers, and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubility, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet and Samuel H. Wilen, “Enantiomers, Racemates and Resolutions,” John Wiley And Sons, Inc., 1981, which is incorporated herein by reference.

The compounds of the invention may be prepared as solvates (e.g., hydrates). The term “solvate” refers to a complex of variable stoichiometry formed by a solute (for example, a compound of the invention or a pharmaceutically acceptable salt thereof) and a solvent. Such solvents for the purpose of the invention may not interfere with the biological activity of the solute. Non-limiting examples of suitable solvents include water, acetone, methanol, ethanol and acetic acid. Preferably the solvent used is a pharmaceutically acceptable solvent.

Furthermore, the compounds of the invention may be prepared as crystalline forms. The crystalline forms may exist as polymorphs.

It should be noted that in view of the close relationship between compound of the invention and their other forms, whenever a compound is referred to in this context herein, a corresponding salt, diastereomer, enantiomer, racemate, crystalline, polymorph, prodrug, hydrate, or solvate is also intended, if it is possible or appropriate under certain circumstances.

As used herein, the term “composition” is intended to encompass a product comprising the claimed compound, salt, diastereomer, enantiomer, racemate, hydrate, solvate, or a pharmaceutical combination thereof in the therapeutically effective amount, as well as any other product which results, directly or indirectly, from claimed compound, salt, diastereomer, enantiomer, racemate, hydrate, solvate, or a pharmaceutical combination thereof. The composition may be formed as a pharmaceutical composition, a food composition, a food additive composition, a feed composition, or a feed additive composition by using appropriate methods known in the art.

As used herein, the term “pharmaceutical composition” refers to a mixture of a therapeutically active component (ingredient) with one or more other components, which may be chemically or biologically active or inactive. Such components may include, but not limited to, carriers, stabilizers, diluents, dispersing agents, suspending agents, thickening agents, excipients, and adjuvants.

As used herein, the term “pharmaceutical combination” means a product that results from the mixing or combining of more than one therapeutically active ingredient.

As used herein, the term “acceptable” with respect to a formulation, composition or ingredient, as used herein, means having no persistent detrimental effect on the general health of the subject being treated.

As used herein, the term “carrier” refers to chemical or biological material that can facilitate the incorporation of a therapeutically active ingredient(s) into cells or tissues.

Suitable excipients may include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone), and lubricants (e.g., magnesium stearate, talc, stearic acid and sodium lauryl sulphate).

Any suitable pharmaceutically acceptable carriers, stabilizers, diluents, dispersing agents, suspending agents, thickening agents, excipients, and adjuvants known to those of ordinary skill in the art for use in pharmaceutical compositions may be selected and employed in the compositions described herein. The compositions described herein may be in the form of a solid, liquid, or gas (aerosol). For example, they may be in the form of tablets (coated tablets) made of, for example, collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another), and aerosols (propellant-containing or -free inhale solutions). Also, the compositions described herein may be formulated for sustained or slow release.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fishes and the like.

As used herein, the term “administration” or “administering” of the subject compound refers to providing a compound of the invention and/or a prodrug thereof to a subject in need of treatment.

As used herein, the term “contacting with” or “applying to” of an object refers to methods of allowing the compositions of the invention to be in contact with or be applied to an object by, for example, wiping, dipping, immersing, or spraying.

As used herein, the term “effective amount” or “therapeutically effective amount” refer to a sufficient amount of an active ingredient(s) described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. By way of example only, a therapeutically effective amount of a compound of the invention may be in the range of e.g., about 0.01 mg/kg/day to about 1000 mg/kg/day, from about 0.1 mg/kg/day to about 500 mg/kg/day, from about 0.1 mg (x2)/kg/day to about 500 mg (x2)/kg/day.

In addition, such compounds and compositions may be administered singly or in combination with one or more additional therapeutic agents. The methods of administration of such compounds and compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration. Compounds provided herein may be administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like. In some embodiments, such pharmaceutical compositions are formulated as tablets, pills, capsules, a liquid, an inhalant, a nasal spray solution, a suppository, a solution, a gel, an emulsion, an ointment, eye drops, or ear drops.

The therapeutically effective amount may vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

The term “metabolic disorder”, as used herein, refers to any disorders caused by metabolic abnormality in lipids or glucose, and includes, but not limited to, obesity, type 2 diabetes, disturbed glucose tolerance, insulin resistance, hyperglycemia, hyperlipidemia, hypertriglyceridemia, hypercholesteremia, and dyslipidemia.

The term “therapeutically effective amount” refers to an amount of a compound of the present invention that ameliorates, attenuates or eliminates a particular disease or condition or prevents or delays the onset of a particular disease or condition. In the case of diabetes mellitus, the therapeutically effective amount of the drug may reduce postprandial blood glucose level; reduce HbA1c level; treat or inhibit diabetic retinopathy or nephropathy; inhibit (slow to some extent and preferably stop) progress of diabetes; weight loss; ameliorate or enhance pancreatic β-cell function; and/or relieve to some extent one or more of the symptoms associated with diabetes. To the extent the drug may modulate blood glucose level to normal state.

The “pharmaceutical composition” as used herein may contain effective component and pharmaceutically acceptable formulation, and the pharmaceutical compositions suitable for the delivery of compounds of the present invention and methods for their preparation can be readily apparent to those skilled in the art.

The pharmaceutical composition as used herein can be administered either orally or parenterally through diverse formulations, and the effective dose of administration may vary depending on physical condition, body weight and severity of the illness of the subject, formulation, and administration time and route, but can be readily determined by those skilled in the art.

Formulations suitable for oral administration include tablets, pills, solid/soft capsules, liquid, suspension, emulsifier, syrups, granules, and elixir, and typically comprise diluents (i.e. lactose, dextrose, sucrose mannitol, solbitol, cellulose, and/or glycine) and lubricant (i.e. silica, talc, stearic acid and its magnesium or calcium salt and/or polyethylene glycol). Tablets of the formulation also comprise binders including magnesium aluminium silicate, starch paste, gelatin, methyl cellulose, sodium carboxy methyl cellulose, and/or poly-venyl pyrrolidine, and depending on circumstances, tablets may comprise disintegrants including starch, agar, alginic acid or its sodium salt, or boiling mixture, absorbent, coloring agent, flavoring agents, or sweeteners.

The pharmaceutical composition as used herein is administered with pharmaceutically effective amounts. The term “pharmaceutically effective amount” refers to sufficient amount of a compound in the present invention that can treat disease with rational and adequate benefit/risk ratio, and the effective amount can be readily determined depending on the types of subject's illness, severity, activity of the compound, sensitivity of the subject to the compound, administration time, route and excretion ratio, treatment interval, factors including co-administered drugs and other well-known medical factors. Compounds of the present invention can be combined or co-administered with other types of drugs as a combination therapy or monotherapy, and can be administered with add-on therapy to the pre-existing treatment with single or multiple administration. Aforementioned factors must be all considered adequately to determine within the bounds of goal achieving maximum therapeutic effect with minimum amounts of the compound without harmful or serious adverse effects, and this process can be readily determined by those skilled in the art.

In particular, the therapeutically effective amount of the compound in the present invention can vary depending on the subject's age, gender, and body weight, and typically ranges from 0.001 to 150 mg per 1 kilogram of body weight, desirably from 0.01 to 100 mg/kg/day or 0.01 to 100 mg/kg/48 hrs with Q.D., B.I.D. or T.I.D.

Other embodiments and uses will be apparent to one skilled in the art in light of the present disclosures. The following examples are provided merely as illustrative of various embodiments and shall not be construed to limit the invention in any way.

### EXAMPLES

The present invention is further exemplified by the following examples. The examples are for illustrative purpose only and are not intended to limit the invention, nor should they be construed as limiting the invention in any manner. Those skilled in the art will appreciate that variations and modifications can be made without changing the scope of the invention.

### Example 1

### Preparation of Crystalline Form of Compound 1

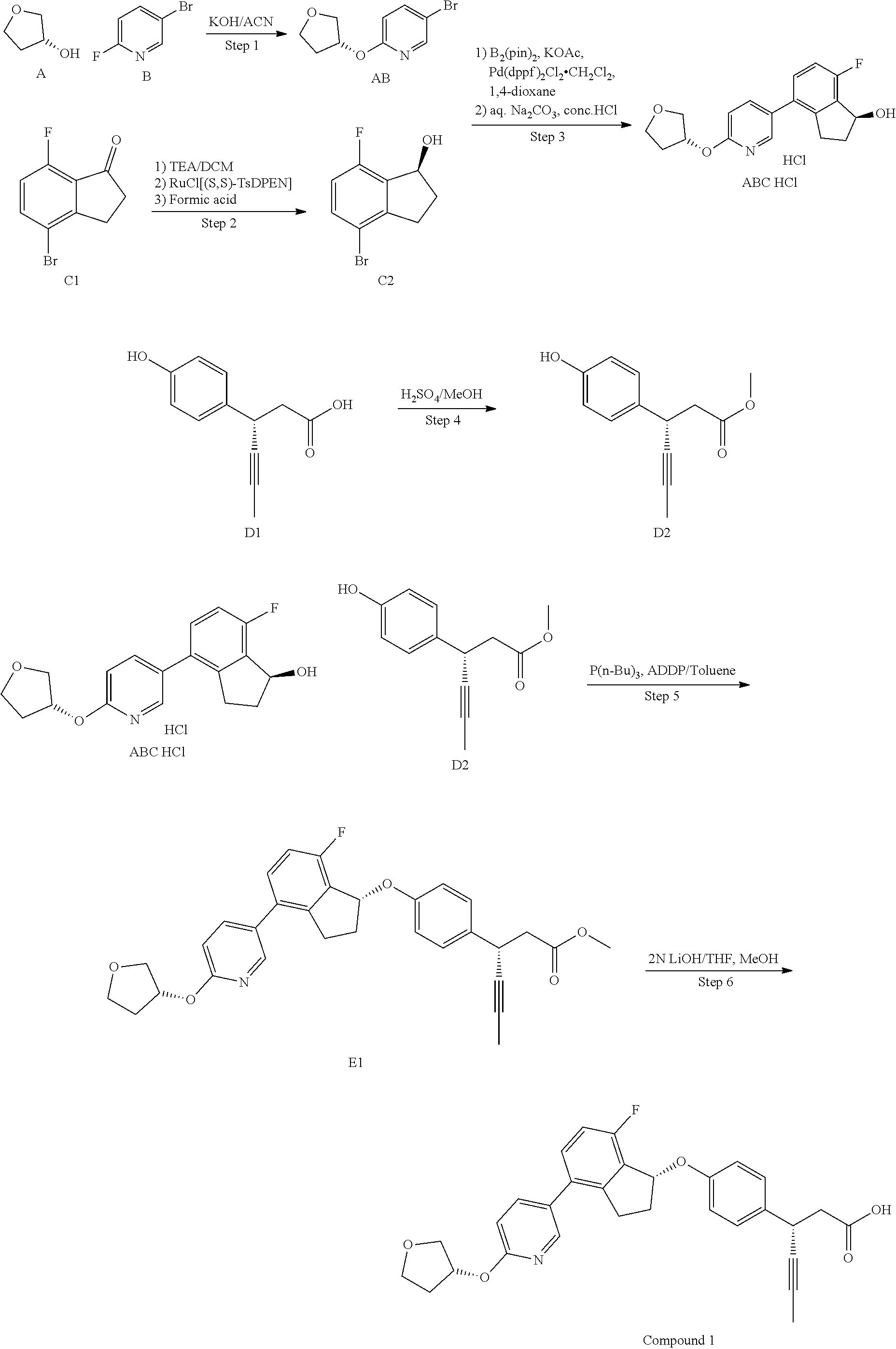

Step 1

Compound A (525.7 g, 1.05 eq.), Compound B (1.0 kg, 1.0 eq.), and acetonitrile (7.0 L) were charged to a 20 L vessel, and potassium hydroxide (478.2 g, 1.5 eq.) was added to the mixture. The reaction mixture was refluxed for 1 hour while the stirred vessel contents were maintained at 90° C. The mixture was then cooled, and then water (1.0 L) was added. The reaction mixture was evaporated at reduced pressure to remove solvent while the stirred vessel contents were maintained at 50° C. When the residue was almost completely concentrated, ethyl acetate (1.0 L) was added to the residue. The resulting mixture was stirred for 10 minutes, and the resulting mixture was evaporated at reduced pressure to remove solvent while the stirred vessel contents were maintained at 50° C. Ethyl acetate (10.0 L) and water (10.0 L) were added to the residue after the resulting mixture was stirred for 10 minutes and then allowed to settle for 5 minutes. The lower (water) phase was removed, and water (5.0 L) was added to the organic phase. The resulting mixture was stirred for 10 minutes, and then allowed to settle for 5 minutes. The lower (water) phase was removed, and saturated brine (5.0 L) and water (5.0 L) were added to the organic phase. The resulting mixture was stirred for 10 minutes and then allowed to settle for 5 minutes. The lower phase was drained to waste disposal. Anhydrous magnesium sulfate (400.0 g) was added to the organic phase. The resulting mixture was stirred at 20˜30° C. for 30 minutes and filtered to remove magnesium sulfate, and filter was washed with ethyl acetate. The filtrate was evaporated at reduced pressure to remove solvent while the stirred vessel contents were maintained at 50° C. When the residue was almost completely concentrated, acetone (2.0 L) was added to the residue. The resulting mixture was stirred for 10 minutes and was evaporated at reduced pressure to remove solvent while the stirred vessel contents were maintained at 50° C. Ethanol (2.8 L) was added when the residue was almost concentrated. The resulting mixture is dissolved while the stirred vessel contents are maintained at 50° C. The mixture was cooled to 30° C., and then water (6.9 L) was added dropwise to the solution for 1 hour. After the generated crystals were aged for 1 hour at room temperature, the crystals were filtered and washed with water. The crystals were dried at 50° C. for 20 hours to obtain dry Compound AB (1.1 kg, yield: 81.0%, white to almost white crystalline powder).

In order to obtain the remaining solid Compound AB that was not discharged from the vessel, acetone (1.0 L) was added to the 20 L vessel and dissolved the solid Compound AB. The mixture was transferred round bottom flask and evaporated at reduced pressure to remove solvent at 50° C. Ethanol (0.2 L) was added when the residue was almost concentrated. The resulting mixture was dissolved at 50° C. The mixture was cooled to 30° C., and then water (0.5 L) was added dropwise to the solution for 15 minutes. After the generated crystals were aged for 20 hours at room temperature, the crystals were filtered and washed with water. The crystals were dried at 50° C. for 20 hours to obtain dry Compound AB. (89.2 g, white to almost white crystalline powder) As a result, Compound AB of 1.2 kg was obtained as yield 87.4%.

Step 2

Compound C1 (500.3 g, 1.0 eq.) and dichloromethane (1.5 L) were charged to a 20 L vessel, and the mixture was cooled to −5° C. Triethylamine (920.0 mL, 3.0 eq.) was added to the mixture, and [(S,S)-N-(2-Amino-1,2-diphenylethyl)-p-toluenesulfonamide]chloro(mesitylene)ruthenium(II) (1.4 g, 0.001 eq.) was added to the mixture at −10° C. Formic acid (290.0 mL, 3.5 eq.) was slowly added to the vessel for 30 minutes.

The reaction mixture was refluxed for 2 hours at 65° C. After cooling the temperature to room temperature, reaction completion was checked by TLC. (Ethyl acetate: n-Hexane=1:5) The reaction mixture was evaporated at reduced pressure to remove solvent while the stirred vessel contents were maintained at 40° C. and then cooled to 25° C. Ethyl acetate (3.0 L) and water (3.0 L) were added to the residue. The resulting mixture was stirred for 10 minutes and then allowed to settle for 10 minutes. The lower (water) phase was removed, and 1N hydrochloride solution (3.0 L) was added to the organic phase. The resulting mixture was stirred for 15 minutes and then allowed to settle for 10 minutes. The lower (water) phase was removed, and 1N sodium bicarbonate solution (3.0 L) was added to the organic phase. The resulting mixture was stirred for 15 minutes and then allowed to settle for 15 minutes. The lower (water) phase was removed, and 10% sodium chloride solution (3.0 L) was added to the organic phase. The resulting mixture was stirred for 15 minutes and then allowed to settle for 15 minutes. The lower phase was drained to waste disposal. Anhydrous magnesium sulfate (211.0 g) and active C (152.3 g) were added to the organic phase.

The resulting mixture was stirred at room temperature for more than 15 hours and filtered through a celite pad to remove magnesium sulfate and active C. The vessel and filter were washed with ethyl acetate.

The filtrate was transferred to the round bottom flask, and it was concentrated using a rotary evaporator. Bath temperature was maintained at 35° C. When the residue was almost completely concentrated, n-hexane (2.5 L) was added and re-evaporated. n-Hexane (2.5 L) was added when the residue was almost concentrated. The resulting mixture was refluxed to dissolve the residue. When the solid was completely dissolved, the round bottom flask was cooled to room temperature. After the generated crystals were aged for 2 hours at room temperature, the crystals were filtered and washed with n-hexane (1.0 L). The crystals were dried at 50° C. for more than 15 hours to obtain dry Compound C2 (377.1 g, yield: 74.7%, white to light gray crystals).

Compound C1 (1.0 kg, 1.0 eq.) and dichloromethane (3.0 L) were charged to a 20 L vessel, and the mixture was then cooled to −10° C. Triethylamine (1.83 L, 3.0 eq.) was added to the mixture and then stirred for 5 minutes. [(S,S)-N-(2-Amino-1,2-diphenylethyl)-p-toluenesulfonamide]chloro (mesitylene)ruthenium(II) (3.0 g, 0.001 eq.) was added to the mixture and then stirred for 5 minutes. Formic acid (580.0 mL, 3.5 eq.) was slowly added to the vessel for 40 minutes at −10° C. The reaction mixture was refluxed for 2 hours at 65° C. After cooling the temperature to room temperature, reaction completion was checked by TLC (Ethyl acetate: n-Hexane=1:5).

The reaction mixture was evaporated at reduced pressure to remove solvent while the stirred vessel contents were maintained at 40° C. and then cooled to 25° C. Ethyl acetate (6.0 L) and water (6.0 L) were added to the residue after the resulting mixture was stirred for 10 minutes and then allowed to settle for 10 minutes. The lower (water) phase was removed, and 1N hydrochloride solution (6.0 L) was added to the organic phase. The resulting mixture was stirred for 15 minutes and then allowed to settle for 10 minutes. The lower (water) phase was removed, and 1N sodium bicarbonate solution (6.0 L) was added to the organic phase. The resulting mixture was stirred for 15 minutes and then allowed to settle for 10 minutes. The lower (water) phase was removed, and 10% sodium chloride solution (6.0 L) was added to the organic phase. The resulting mixture was stirred for 15 minutes and then allowed to settle for 10 minutes. The lower phase was drained to waste disposal. Anhydrous magnesium sulfate (420.0 g) and active C (300.0 g) were added to the organic phase. The resulting mixture was stirred at room temperature for more than 15 hours and filtered through a celite pad to remove magnesium sulfate and active C from the vessel. The filtrate was evaporated at reduced pressure to remove solvent while the stirred vessel contents were maintained at 50° C. or below.

When the residue reached almost 2.0 L, n-hexane (5.0 L) was added and re-evaporated. When the residue reached almost 3.0 L, the concentration was stopped. n-Hexane (2.0 L) was added to make the residue 5.0 L. The resulting mixture was refluxed to dissolve the residue at 75° C. When the solid was completely dissolved, the vessel was cooled to 30° C. for 30 minutes. After the generated crystals were aged for 2 hours 30 minutes at room temperature, the crystals were filtered and washed with n-hexane (2.0 L). The crystals were dried at 50° C. for more than 15 hours to obtain dry Compound C2 (576 .9 g, yield: 57.0%, White to light gray crystals).

The filtrate was transferred to the round bottom flask and concentrated at 40° C. When the residue was almost completely concentrated, n-hexane was added and re-evaporated. n-Hexane (1.2 L) was added to the residue and heated to completely dissolve the solid and cool the solution to room temperature. After the generated crystals were aged for more than 15 hours at room temperature, the crystals were filtered and washed with n-hexane. The crystals were dried at 50° C. for more than 15 hours to obtain dry Compound C2 (190.0 g, White to light gray crystals). As a result, Compound C2 of 766.9 g is obtained as yield 75.0%.

Step 3

Compound AB (1000.4 g, 1.0 eq.) and 1,4-dioxane (10.0 L) were charged to a vessel and bis(pinacolato)diboron (1144.1 g, 1.1 eq.), potassium acetate (804.2 g, 2.0 eq.) and 1,1′-bis(diphenylphosphino)ferrocene-palladium(II) dichloride dichloromethane complex (167.1 g, 0.05 eq.) were added to the mixture. The reaction mixture was then refluxed for 2 hours. Reaction completion was checked by TLC (Ethyl acetate: n-Hexane=1:2), and then the mixture was cooled to 70° C. Compound C2 (947.1 g, 1.0 eq.). Aq. sodium carbonate solution (868.0 g of Na2CO3, 2.0 eq. +10.0 L of H2O) was added. The reaction mixture was refluxed for 2 hours. The jacket temperature was cooled step by step (at 80° C. for 20 min, at 60° C. for 30 min, at 40° C. for 20 min), and the reaction mixture was stirred at 25° C. for one day. Reaction completion was checked by TLC (Ethyl acetate: n-Hexane=1:1).

Ethyl acetate (20.0 L) was added to reaction mixture and stirred for 30 minutes. The resulting mixture was filtered through a celite pad. The filtrate was allowed to settle for 10 minutes, and then the lower (water) phase was removed. 10% Sodium chloride solution (20.0 L) was added to the organic phase, and the resulting mixture was stirred for 30 minutes and then allowed to settle for 20 minutes. But there was no phase separation, so additional 10% sodium chloride solution (5.0 L) was added. The resulting mixture was stirred for 5 minutes and then allowed to settle for 30 minutes. The lower (water) phase was removed, and anhydrous magnesium sulfate (2.0 kg) and active C (1.0 kg) were added to the organic phase. The resulting mixture was stirred at 20˜30° C. for one day and filtered through a celite pad to remove magnesium sulfate and active C.

The filtrate was evaporated at reduced pressure to remove solvent while the stirred vessel contents were maintained at 40° C. or below. When the residue was almost completely concentrated, methanol (3.0 L) was added and re-evaporated. Methanol (1.0 L) and acetone (3.0 L) were added to the residue to dissolve residue. Conc. hydrochloride (355.6 mL, 1.0 eq.) was added for 3 minutes, and acetone (4.0 L) was added. After the generated crystals were aged for 2 hours at room temperature, the crystals were filtered and washed with acetone (6.0 L). The crystals were dried at 50° C. for one day to obtain dry Compound ABC HCl (1.1 kg, yield: 76.7%, white to almost white crystalline powder).

Step 4

Compound D1 (418.0 g, 1.0 eq.) and methanol (4.2 L) were charged to a vessel, and sulfuric acid (10.9 mL, 0.1 eq.) was added to the mixture. The reaction mixture was then refluxed for 4 hours. Reaction completion was checked by TLC (Ethyl acetate: n-Hexane=1:1), and then the mixture was cooled to 40° C.

Reaction mixture was sampled for HPLC analysis, and it was evaporated at reduced pressure to remove solvent while the stirred vessel contents were maintained at 40° C. or below. When the residue was almost completely concentrated, ethyl acetate (1.3 L) was added and re-evaporated. Ethyl acetate (4.2 L) and saturated sodium bicarbonate solution (4.2 L) were added to the residue after the resulting mixture was stirred for 10 minutes and then allowed to settle for 5 minutes.

The lower (water) phase was removed such that there was no product in water layer with TLC. Water (4.2 L) was added to the organic phase, and the resulting mixture was stirred for 10 minutes and then allowed to settle for 5 minutes. The lower (water) phase was removed such that there was no product in water layer with TLC. 10% Sodium chloride solution (4.2 L) was added to the organic phase, and the resulting mixture was stirred for 10 minutes and then allowed to settle for 5 minutes. The lower phase was drained to waste disposal. Anhydrous magnesium sulfate (292.6 g) was added to the organic phase, and the resulting mixture was stirred at 20˜30° C. for 30 minutes and filtered to remove magnesium sulfate. Filter was washed with ethyl acetate. The filtrate was sampled for HPLC analysis and was maintained to settle for one day.

The filtrate was evaporated at reduced pressure to remove solvent while the stirred vessel contents were maintained at 40° C. or below. When the residue was almost completely concentrated, toluene (1.3 L) was added to the residue and re-evaporated at reduced pressure to remove solvent while the stirred vessel contents were maintained at 40° C. or below. The resulting residue was sampled for HPLC analysis (oil form).

Step 5

Compound ABC HCl (600.2 g, 1.0 eq.), dichloromethane (6.0 L) and water (6.0 L) were charged to a vessel and stirred. 1N Sodium hydroxide solution was added to the mixture, and it was titrated to pH 12˜13 (by using about 3.0 L of 1N sodium hydroxide solution). After organic phase was separated, water phase was checked by TLC. Product was remained in the water phase, so dichloromethane (3.0 L) was added to water phase and re-extracted such that there was no product in water layer with TLC. 10% Sodium chloride solution (6.0 L) was added to the combined organic phase, and the upper phase was removed. Anhydrous magnesium sulfate (420.0 g) was added to the organic phase, and the resulting mixture was stirred at 20˜30° C. for 30 minutes and filtered to remove magnesium sulfate. Filter was washed with dichloromethane. The filtrate was evaporated at reduced pressure to remove solvent while the stirred vessel contents were maintained at 40° C. or below. When the residue was almost completely concentrated, toluene (1.8 L) was added to the residue and re-evaporated at reduced pressure to remove solvent while the stirred vessel contents were maintained at 40° C. or below. The resulting residue was sampled for HPLC analysis, and it was diluted with toluene (5.0 L).

Compound ABC diluted with 5 L of toluene, D2 diluted with 5 L of toluene, and toluene (8.0 L) were charged to a vessel. The mixture was then cooled while the stirred vessel contents were maintained at 15° C. or below. Tri-n-butyl phosphine (553.8 mL, 1.3 eq.) was added to the mixture, and 1,1′-(Azodicarbonyl)dipiperidine (559.4 g, 1.3 eq.) was slowly added while the stirred vessel contents were maintained at 12˜16° C. The reaction mixture was stirred at 20˜30° C. for one day. Reaction mixture was sampled for HPLC analysis, and n-hexane (12.0 L) was added to the resulting mixture. The mixture was stirred for 30 minutes and filtered out by-product. The filtrate was evaporated at reduced pressure to remove solvent while the stirred vessel contents were maintained at 40° C. or below. When the residue was almost completely concentrated, n-hexane (2.4 L) was added to the residue and re-evaporated at reduced pressure to remove solvent while the stirred vessel contents were maintained at 40° C. or below. Dichloromethane (900.0 mL) and n-hexane (2.4 L) were added when the residue was almost concentrated. The resulting mixture was refluxed to dissolve the residue. When the reactants were dissolved, the vessel was cooled to 25˜30° C. After the generated crystals were aged for one day at room temperature, the crystals were filtered and washed with n-hexane. The crystals were dried at 50° C. for 24 hours to obtain dry Compound E1 (594.2 g, yield: 67.6%, off-white crystal powder).

In order to obtain product from the residue, the residue was evaporated at reduced pressure. n-Hexane (2.0 L) was added when the residue was almost concentrated and stirred at 25˜30° C. for 1 hour. Crude product was filtered and dried at 50° C. for 4 hours. Dichloromethane (180.0 mL) and n-hexane (480.0 mL) were added to the crude product (119.7 g), and the mixture was refluxed to dissolve the crude product. After that, the vessel was cooled to 25˜30° C., and the generated crystals were aged for one day at 20˜30° C. The crystals were filtered and washed with n-hexane. The crystals were dried at 50° C. for 24 hours to obtain dry Compound E1 (104.5 g, Off-white crystal powder). As a result, Compound E1 of 698.7 g was obtained as yield 79.4%.

Step 6

Compound E1 (90.0 g), tetrahydrofuran (1.44 L), and methanol (0.36 L) were added to a 3 L round flask, and the mixture was cooled to 10° C. 2.0 N lithium hydroxide solution (43.95 g of lithium hydroxide monohydrate 6.0 eq.+0.52 L of water) was added for 5 minutes. The mixture was stirred for 3 hours at 20˜30° C. It was confirmed by TLC (EA: n-Hex=1:1) that the reaction was completed. The mixture was cooled to 10° C. 1N hydrochloric acid solution was added. The mixture was titrated to pH 2.0˜3.0. The mixture was heated at 20˜30° C. 0.9 L of ethyl acetate was added. The mixture was stirred for 30 minutes and allowed to settle for 15 minutes. The lower phase was removed, and 0.45 L of ethyl acetate was added. The mixture was stirred for 20 minutes and allowed to settle for 15 minutes. The lower phase was removed. 0.9 L of 10% sodium chloride solution was added to the combined organic phase. The mixture was stirred for 15 minutes and allowed to settle for 10 minutes. The lower phase was removed, and the organic phase was allowed to settle for 1 day. Anhydrous magnesium sulfate (300 g) was added to the organic phase. The mixture was stirred at 20˜30° C. for 30 minutes. Magnesium sulfate was filtered out. The filtrate was washed with ethyl acetate. The filtrate was evaporated at reduced pressure to remove the solvent while the residue was maintained at 40° C. or below. When the residue was almost completely concentrated, 0.27 L of acetone was added. It was re-evaporated at reduced pressure to remove the solvent while the residue was maintained at 40° C. or below. When the residue was almost completely concentrated, 0.27 L of acetone was added. The resulting mixture was refluxed to dissolve the residue. When the reactants were dissolved, the flask was cooled to 40° C., and 0.54 L of n-heptane was added to the mixture. It was stirred for 15˜20 hours at 20˜30° C. The resulting crystals were filtered and dried at 50° C. for 15˜20 hours to obtain crystalline form Compound 1 (74.4 g, yield: 85.0%).

### Example 2

### X-Ray Powder Diffraction Pattern Analysis

The crystalline form Compound 1 prepared according to Example 1 was characterized by X-ray powder diffraction (XRPD) pattern. The XRPD spectra were collected with X'celerator detector using a standard Aptuit method. The data were evaluated using the HighScore Plus or the Data Viewer software. The instrumental parameters used are listed below.

| Instrumental | |
|---|---|
| parameter | Value |
| 2-theta range | 2-45 |
| Step size [°2-theta] | 0.0170 |
| Time per step [sec] | 60.7285 sec |
| Wavelength [nm] | 0.154060 (Cu K-Alpha 1) |
| Rotation [Yes/No] | Yes |
| Slits divergence/ | Incident Mask fixed 10 mm; Divergence slits ½, |
| antiscatter | Antiscat. slits ½ on incident beam; 1/32 on diffracted |
| X-ray Mirror | Inc. Beam Cu W/Si focusing MPD, |
| | Acceptance Angle 0.8°, Length 55.3 mm |
| Temperature | Room temperature |
| Humidity values | Ambient |
| [% RH] | |
| Fixed Slits | 0.02 rad fixed Soller slits on incident and diffracted beam |
| Monochromator | None |
| Detector type | X'celerator (active length 2.122 2theta degree), scanning |
| | mode |
| Sample holder | Transmission sample holder. Use Insert to keep |
| | thickness at 1 mm, typically 5 mm diameter |
| Configuration | Transmission |
| Generator | 40 KV/40 mA |
| voltage/current | |

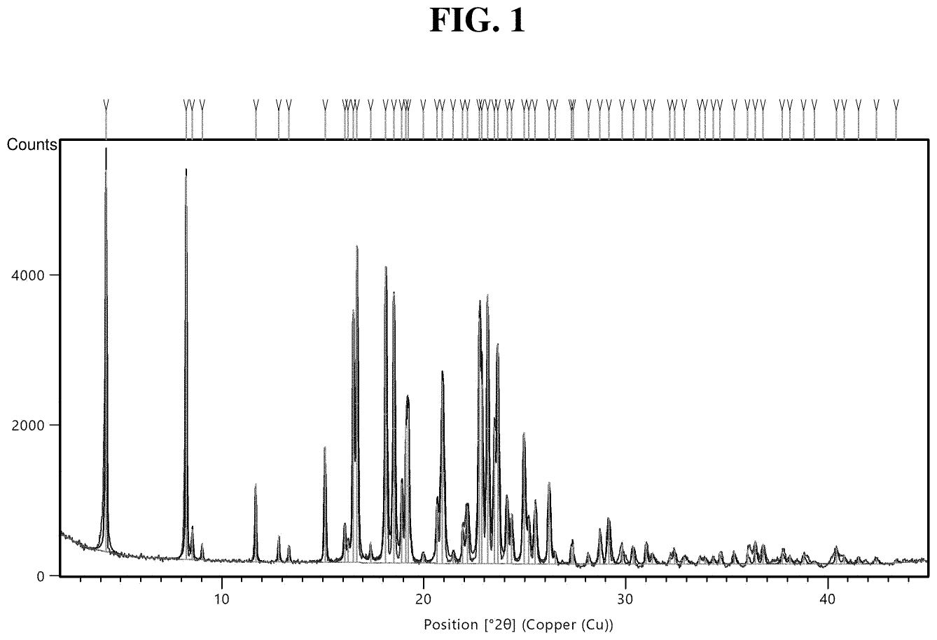

FIG. 1 and Table 1 show an X-ray powder diffraction (XRPD) pattern of the crystalline form Compound 1 prepared according to Example 1. As shown in FIG. 1 and Table 1, the XRPD pattern includes five or more peaks with degrees two-theta positions selected from the group consisting of the peak positions five or more peaks with degrees two-theta positions selected from the group consisting of 4.2721, 8.2390, 8.5441, 9.0268, 11.6804, 12.8279, 13.3227, 15.1104, 16.0874, 16.2609, 16.5082, 16.6950, 17.3649, 18.1220, 18.5187, 18.9074, 19.1174, 19.2305, 19.9625, 20.6590, 20.9169, 21.4576, 21.9189, 22.1679, 22.7467, 22.8806, 23.1588, 23.4796, 23.6577, 24.1282, 24.3485, 24.9581, 25.1935, 25.5077, 26.2001, 26.5042, 27.2953, 27.3795, 28.1568, 28.7195, 29.1545, 29.8201, 30.3630, 30.9912, 31.3151, 32.1743, 32.4060, 32.8746, 33.6575, 33.9219, 34.3338, 34.6532, 35.3607, 36.0311, 36.4072, 36.7779, 37.7329, 38.1170, 38.7922, 39.3418, 40.4170, 40.8055, 41.5236, 42.3929, and 43.3903, wherein each peak of degree two-theta has a margin of error of ±0.2.

TABLE 1

| Pos. [°2θ] | Height [cts] | FWHM Left [°2θ] | d-spacing [Å] | Rel. Int. [%] |
|---|---|---|---|---|
| 4.2721 | 5181.32 | 0.0836 | 20.68384 | 99.53 |
| 8.2390 | 5205.58 | 0.0669 | 10.73173 | 100.00 |
| 8.5441 | 406.42 | 0.0669 | 10.34919 | 7.81 |
| 9.0268 | 215.32 | 0.0669 | 9.79688 | 4.14 |
| 11.6804 | 1053.27 | 0.0669 | 7.57645 | 20.23 |
| 12.8279 | 352.48 | 0.0669 | 6.90121 | 6.77 |
| 13.3227 | 210.35 | 0.0669 | 6.64598 | 4.04 |
| 15.1104 | 1561.59 | 0.0836 | 5.86346 | 30.00 |
| 16.0874 | 512.08 | 0.1004 | 5.50953 | 9.84 |
| 16.2609 | 285.10 | 0.0502 | 5.45111 | 5.48 |
| 16.5082 | 3423.19 | 0.0836 | 5.37002 | 65.76 |
| 16.6950 | 4302.21 | 0.1004 | 5.31034 | 82.65 |
| 17.3649 | 268.39 | 0.0669 | 5.10697 | 5.16 |
| 18.1220 | 4019.65 | 0.1171 | 4.89526 | 77.22 |
| 18.5187 | 3653.99 | 0.1171 | 4.79130 | 70.19 |
| 18.9074 | 1074.83 | 0.1004 | 4.69367 | 20.65 |
| 19.1174 | 1866.22 | 0.0836 | 4.64258 | 35.85 |
| 19.2305 | 2094.95 | 0.1004 | 4.61553 | 40.24 |
| 19.9625 | 128.44 | 0.1338 | 4.44791 | 2.47 |
| 20.6590 | 800.13 | 0.1004 | 4.29949 | 15.37 |
| 20.9169 | 2469.26 | 0.1840 | 4.24705 | 47.43 |
| 21.4576 | 129.15 | 0.1171 | 4.14124 | 2.48 |
| 21.9189 | 486.49 | 0.0836 | 4.05512 | 9.35 |
| 22.1679 | 769.99 | 0.1506 | 4.01013 | 14.79 |
| 22.7467 | 3330.27 | 0.1338 | 3.90939 | 63.97 |
| 22.8806 | 2430.95 | 0.0669 | 3.88682 | 46.70 |
| 23.1588 | 3653.73 | 0.1171 | 3.84075 | 70.19 |
| 23.4796 | 1846.68 | 0.0836 | 3.78900 | 35.48 |
| 23.6577 | 2968.29 | 0.1171 | 3.76087 | 57.02 |
| 24.1282 | 888.56 | 0.1171 | 3.68859 | 17.07 |
| 24.3485 | 682.45 | 0.1004 | 3.65571 | 13.11 |
| 24.9581 | 1777.84 | 0.1338 | 3.56779 | 34.15 |
| 25.1935 | 613.94 | 0.1004 | 3.53499 | 11.79 |
| 25.5077 | 819.48 | 0.1506 | 3.49214 | 15.74 |
| 26.2001 | 1108.47 | 0.1338 | 3.40141 | 21.29 |
| 26.5042 | 160.46 | 0.0836 | 3.36307 | 3.08 |
| 27.2953 | 239.55 | 0.0816 | 3.26466 | 4.60 |
| 27.3795 | 268.08 | 0.0502 | 3.25751 | 5.15 |
| 28.1568 | 141.93 | 0.1004 | 3.16933 | 2.73 |
| 28.7195 | 436.44 | 0.1171 | 3.10850 | 8.38 |
| 29.1545 | 624.93 | 0.1673 | 3.06311 | 12.00 |
| 29.8201 | 261.62 | 0.0669 | 2.99623 | 5.03 |
| 30.3630 | 236.66 | 0.1673 | 2.94388 | 4.55 |
| 30.9912 | 273.68 | 0.0836 | 2.88563 | 5.26 |
| 31.3151 | 153.26 | 0.1338 | 2.85651 | 2.94 |
| 32.1743 | 107.38 | 0.1004 | 2.78217 | 2.06 |
| 32.4060 | 188.75 | 0.1338 | 2.76280 | 3.63 |
| 32.8746 | 97.68 | 0.2676 | 2.72448 | 1.88 |
| 33.6575 | 99.19 | 0.1338 | 2.66288 | 1.91 |
| 33.9219 | 84.99 | 0.1673 | 2.64273 | 1.63 |
| 34.3338 | 89.10 | 0.1171 | 2.61197 | 1.71 |
| 34.6532 | 173.05 | 0.0836 | 2.58862 | 3.32 |
| 35.3607 | 170.32 | 0.0836 | 2.53843 | 3.27 |
| 36.0311 | 184.68 | 0.0836 | 2.49272 | 3.55 |
| 36.4072 | 301.47 | 0.0836 | 2.46783 | 5.79 |
| 36.7779 | 266.98 | 0.1004 | 2.44380 | 5.13 |
| 37.7329 | 155.78 | 0.1673 | 2.38412 | 2.99 |
| 38.1170 | 99.74 | 0.1338 | 2.36098 | 1.92 |
| 38.7922 | 131.55 | 0.1338 | 2.32142 | 2.53 |
| 39.3418 | 33.05 | 0.1673 | 2.29025 | 0.63 |
| 40.4170 | 209.49 | 0.1338 | 2.23177 | 4.02 |
| 40.8055 | 111.51 | 0.1338 | 2.21142 | 2.14 |
| 41.5236 | 82.35 | 0.1338 | 2.17482 | 1.58 |
| 42.3929 | 66.25 | 0.2007 | 2.13221 | 1.27 |
| 43.3903 | 37.19 | 0.1338 | 2.08548 | 0.71 |

### Example 3

### Thermal Analysis

The crystalline form Compound 1 prepared according to Example 1 was characterized by thermal gravimetric analysis (TGA) and differential scanning calorimetry (DSC). The TGA analysis was made by a TA Q5000 instrument. The DSC analysis was made by a TA Q2000 MDSC instrument. The instrumental parameters used are listed below.

| | |
|---|---|
| Instrumental parameter | Value |

TGA

| | |
|---|---|
| Balance purge gas [mL/min] | 10 |
| Sample purge gas [mL/min] | 25 |
| Gas | Nitrogen |
| Temperature-Time-Rate | Typically from room temperature |
| | to 350° C. at 10° C./min |
| Typical sample amount [mg] | Usually from 2 mg to 20 mg |
| Pan [Pt/Al] | Sealed Aluminum (punched) |

DSC

| | |
|---|---|
| Instrumental parameter | Value |
| Cooling [ON/OFF] | ON |
| Gas | Nitrogen |
| Temperature-Time-Rate | Typically from 0° C. to 250° C. |
| | Ramp at 10° C./min. |
| Typical sample amount [mg] | Usually from 0.5 mg to 2.5 mg |
| Pan | Aluminum |

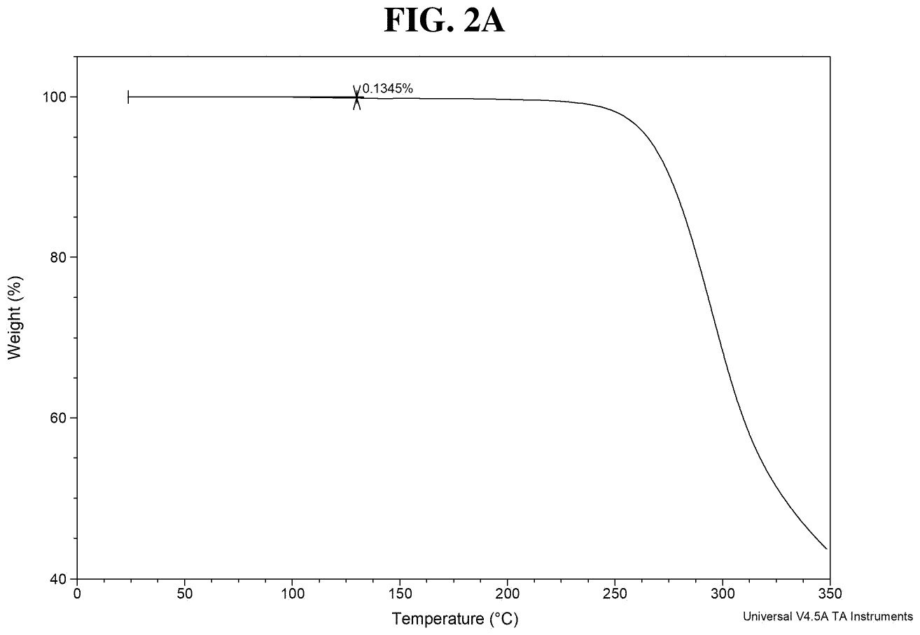

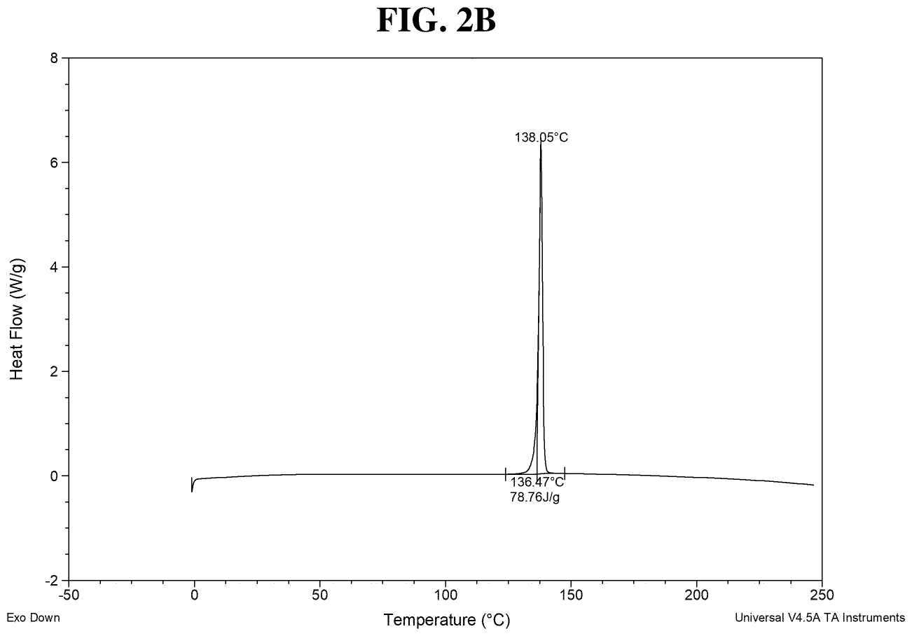

FIGS. 2A and 2B show the results of the TGA and DSC analyses, respectively. The TGA profile showed a weight loss of 0.13% w/w from ambient to 130° C. The DSC trace recorded with a heating rate of 10° C./min showed a small endothermic change in the baseline, followed by a sharp endotherm that corresponds to the melting of the crystalline form at an onset temperature of 136.47° C. and an enthalpy of 78.76 J/g. It was confirmed the crystalline form compound according the present invention has a relatively high thermal stability.

### Example 4

### Polarized Optical Microscopy Analysis

Polarized optical microscopy analysis was made using an Olympus BX41 microscope or a Leica DM microscope equipped with a double polarizer and digital camera. The instrumental parameters used are listed below.

| Instrumental parameter | Value |
|---|---|
| Polarized light [Y/N] | Yes |
| Magnification [eyepiece] | 10x |
| Objective | Typically 5x, 10x, 20x |
| Filter slider | Use the best filter to optimize the image |

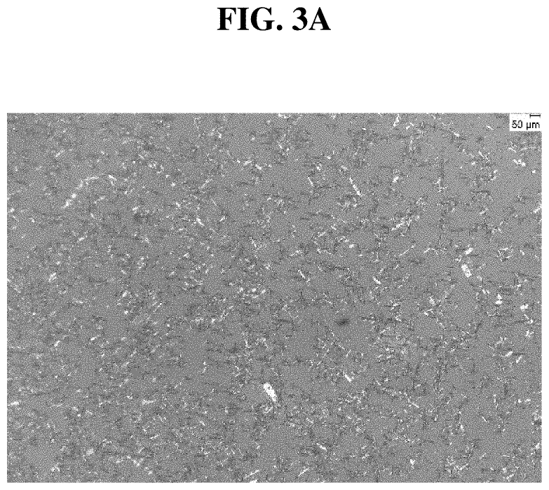

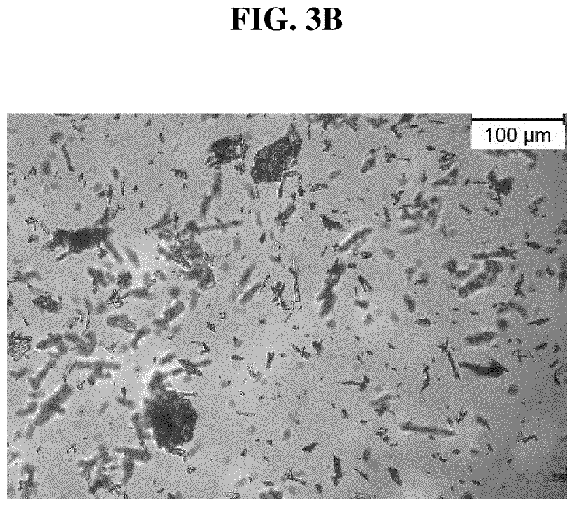

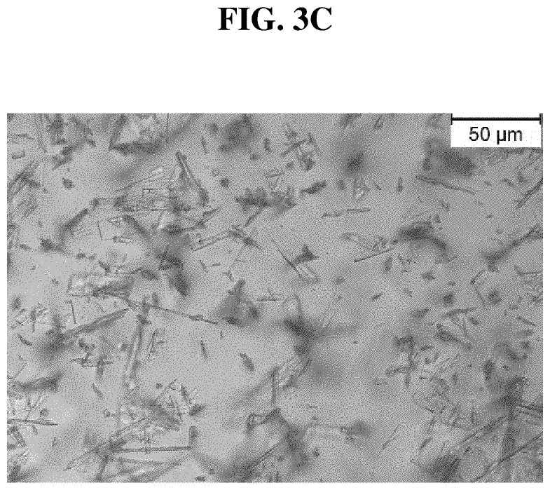

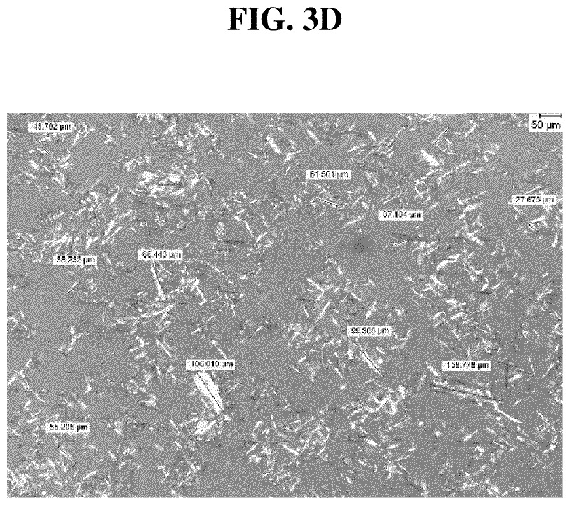

FIGS. 3A to 3D illustrate polarized optical microscopy images of the crystalline form prepared according to Example 1, which were collected at different magnifications. It showed presence of a mixture of acicular and irregular particles with strong birefringence, typically with lengths up to ca. 100 μm and presence of some agglomerates.

### Example 5

### HPLC Analysis

The crystalline form Compound 1 prepared according to Example 1 was characterized by HPLC using a generic method. Instrumental parameters used are listed below.

| Instrumental parameter | Value |
|---|---|
| Column | Phenomenex Luna C18 (50 × 2 mm, 3 μm); |
| | column temperature 40° C. |
| Mobile phase | A: 0.05% TFA/water; B: 0.05% TFA/acetonitrile |
| Gradient | 0 min 100% A to 8 min 95% B |
| Flow [mL/min] | 1.0 |
| Detector | UV at 220 nm |
| Samples preparation | Approximately 1 mg/mL; dissolution in ACN |
| | or ACN/water 8:2 |
| Retention time [min] | ~5.7 |

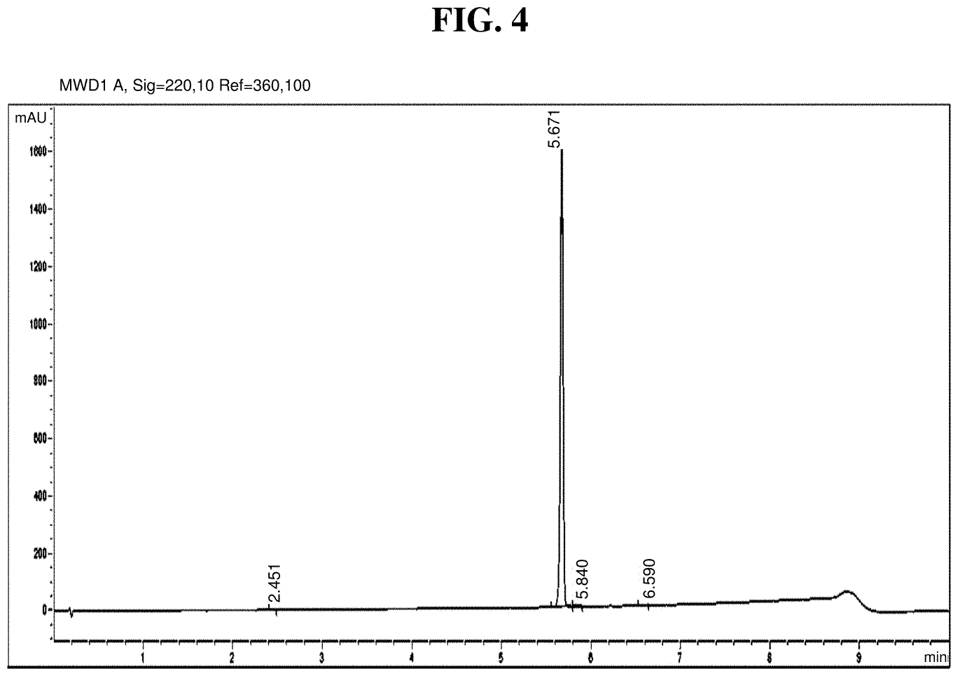

FIG. 4 illustrates the HPLC analysis result, which shows the purity of 99.8% a/a at 220 nm.

### Example 6

### Solution ¹H-NMR Analysis

An appropriate amount of the crystalline form Compound 1 prepared according to Example 1 was dissolved in 0.75 ml of deuterated dimethyl sulfoxide (DMSO-d6) or other deuterated solvent system. All spectra were referenced to the residual solvent line. ¹H NMR spectra were recorded at 25° C. using a Varian INOVA 400 MHz NMR Spectrometer equipped with a Varian ATB probe. The pre-acquisition delay was set to 10 seconds in order to improve relaxation for quantification purposes. Appropriate phasing and baseline corrections were applied in processing the spectra.

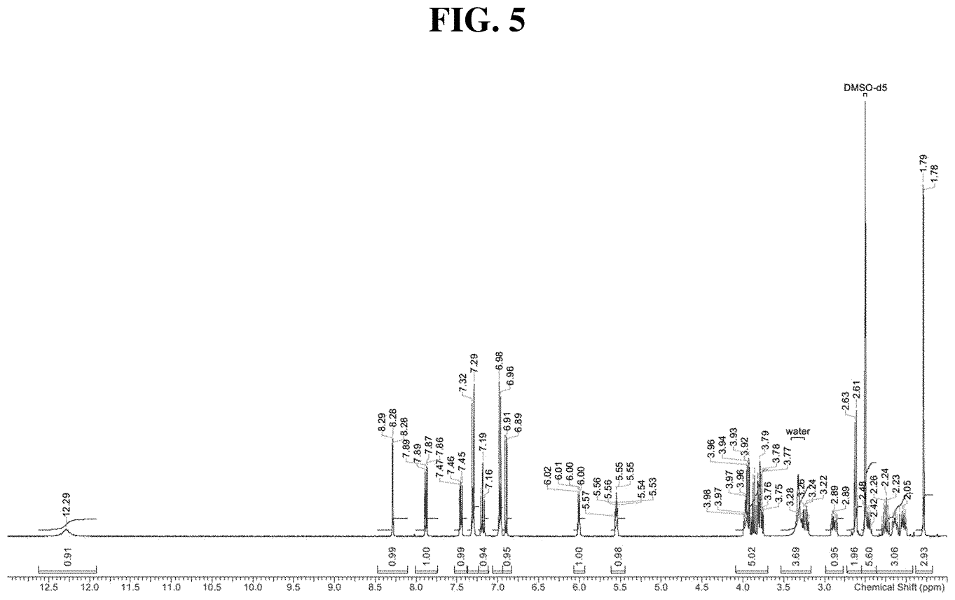

FIG. 5 illustrates the solution 1H-NMR spectrum (DMSO-d6) of the crystalline form Compound 1.

### Example 7

### Gravimetric Vapor Sorption Analysis

The GVS analysis was made using an IGA Sorp instrument by Hiden Analytical. The instrumental parameters are listed below.

| Instrumental parameter | Value |
|---|---|
| Type of analysis | Isotherm |
| Operating temperature [° C.] | 25 |
| Temperature stability | 0.2 |
| [° C./min] | |
| Humidity values [% RH] | Complete Isotherm: 50-60-70-80-90-80-70-60-50-40-30-20- |
| | 10-Dry-10-20-30-40-50-60-70-80-90 |
| Flow rate [mL/min] | 500 |
| Fitting functions | F1 |
| Initial conditions | (1) 25° C., 50% RH, start with adsorption scan during the |
| | complete isotherm. |
| | (2) 25° C., 50% RH, start with desorption scan during the |
| | drying isotherm. |
| End status | End or Keep humidity control |
| Typical sample amount [mg] | Not Less than 10 mg |

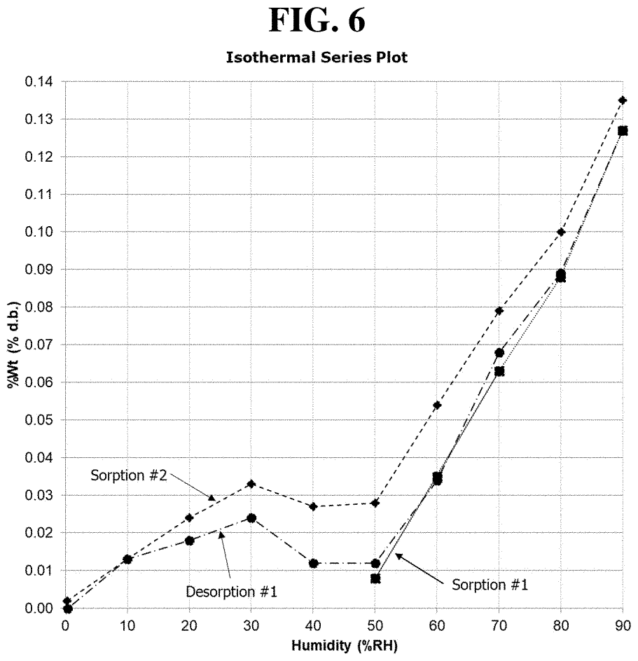

FIG. 6 illustrates the GVS analysis result. Amounts of desorbed/adsorbed water are reported in Table 2. The crystalline form showed a non-hygroscopic behavior (less than 0.2% w/w of weight gain from dryness to 90% RH during the second adsorption cycle).

TABLE 2

| | 1^{st} adsorption | 1^{st} desorption | 2^{nd} adsorption |
|---|---|---|---|
| | cycle | cycle | cycle |
| | (50-90% RH) | (90-0% RH) | (0-90% RH) |
| Name | (% w/w) | (% w/w) | (% w/w) |
| Crystalline Form | 0.119 | 0.127 | 0.135 |
| Compound 1 | | | |

### Example 8

### Solubility Analysis

The solubility of the crystalline form Compound 1 was visually determined in 13 solvents. 5.0±0.2mg of the crystalline form Compound 1 was stirred at 20° C. with increasing volumes of solvents until a clear solution was visually observed. Table 3 shows a summary of visual solubility data at 20° C.

TABLE 3

| Number | Solvent | Solubility (S) (mg/mL) |
|---|---|---|
| 1 | Acetone | S > 100 |
| 2 | Acetonitrile | 6 < S < 8 |
| 3 | 2-Butanone | S > 100 |
| 4 | Dichloromethane | S > 100 |
| 5 | Ethyl acetate | 50 < S < 100 |
| 6 | Methanol | 6 < S < 8 |
| 7 | Methyl tert-butyl ether | 2 < S < 5 |
| 8 | 2-Propanol | 5 < S < 6 |
| 9 | i-Propyl acetate | 17 < S < 25 |
| 10 | Tetrahydrofuran | S > 100 |
| 11 | Toluene | 10 < S <12 |
| 12 | Acetone/water 90:10 | S > 100 |
| 13 | Methanol/water 90:10 | 1 < S < 2 |

### Example 9

### Cell-Based Aequorin Assay

The agonistic activity of the crystalline form of Compound 1 was measured by using the method described in U.S. Patent Application Publication No. 2020/0223833, which is incorporated herein by reference. The agonistic activity of the crystalline form of Compound 1 was higher than 70% of the activity of the reference agonist used in U.S. Patent Application Publication No. 2020/0223833, which falls within the category of the publication.

1. A crystalline form of Compound 1, having an X-ray powder diffraction pattern comprising peaks with degrees two-theta positions of 8.2390, 11.6804, 15.1104, 16.5082 and 20.9169, wherein each peak of degree two-theta has a margin of error of ±0.2.

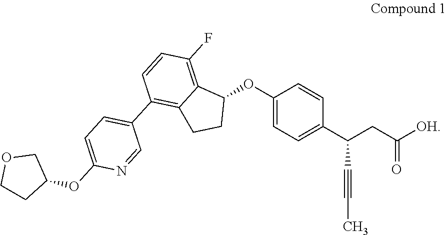 [SMILES: CC#C[C@@H](CC(=O)O)c1ccc(O[C@@H]2CCc3c(-c4ccc(O[C@@H]5CCOC5)nc4)ccc(F)c32)cc1]

2. The crystalline form of claim 1, having an X-ray powder diffraction pattern comprising five or more peaks with degrees two-theta positions selected from the group consisting of 4.2721, 8.2390, 8.5441, 9.0268, 11.6804, 12.8279, 13.3227, 15.1104, 16.0874, 16.2609, 16.5082, 16.6950, 17.3649, 18.1220, 18.5187, 18.9074, 19.1174, 19.2305, 19.9625, 20.6590, 20.9169, 21.4576, 21.9189, 22.1679, 22.7467, 22.8806, 23.1588, 23.4796, 23.6577, 24.1282, 24.3485, 24.9581, 25.1935, 25.5077, 26.2001, 26.5042, 27.2953, 27.3795, 28.1568, 28.7195, 29.1545, 29.8201, 30.3630, 30.9912, 31.3151, 32.1743, 32.4060, 32.8746, 33.6575, 33.9219, 34.3338, 34.6532, 35.3607, 36.0311, 36.4072, 36.7779, 37.7329, 38.1170, 38.7922, 39.3418, 40.4170, 40.8055, 41.5236, 42.3929, and 43.3903, wherein each peak of degree two-theta has a margin of error of ±0.2.

3. The crystalline form of claim 1, having an X-ray powder diffraction pattern that is identical or similar to that of FIG. 1.

4. The crystalline form of claim 1, having a differential scanning calorimetry thermogram exhibiting an endotherm with an onset of about 136° C.

5. The crystalline form of claim 1, having a differential scanning calorimetry thermogram exhibiting an endotherm with an onset of 136.47° C.

6. The crystalline form of claim 1, having a differential scanning calorimetry thermogram that is identical or similar to that of FIG. 2.

7. A solid or semi-solid pharmaceutical dosage form comprising a crystalline form of claim 1 and a pharmaceutically acceptable excipient.

8. A pharmaceutical composition comprising a crystalline form of claim 1 and a pharmaceutically acceptable excipient.

9. A method for treating a disease, disorder, or condition associated with G-protein-coupled receptor 40 (GPR40), the method comprising administering to a subject a therapeutically effective amount of a pharmaceutical composition comprising a crystalline form of claim 1 and a pharmaceutically acceptable excipient.

10. The method of claim 8, wherein the disease, disorder, or condition is selected at least one from the group consisting of obesity, type 2 diabetes, incompatible glucose tolerance, insulin resistance, hyperglycemia, hyperlipidemia, hypertriglyceridemia, and hypercholesterolemia.

11. The method of claim 8, wherein one or more additional active or therapeutic agents are administered to the subject with the pharmaceutical composition.
